(19) United States
(12) Patent Application Publication
(10) Pub. No.: US 2019/0133884 A1
(43) Pub. Date: May 9, 2019

(54) METHOD OF MANAGING MEDICATION OF A USER

(71) Applicant: IoT Instruments Oy, Kuopio (FI)

(72) Inventors: Harri Puolitaival, Jorkirinne (FI); Antti Puolitaival, Hiidenkatu (FI)

(21) Appl. No.: 16/307,292

(22) PCT Filed: May 30, 2017
(86) PCT No.: PCT/FI2017/050401

§ 371 Date: Dec. 5, 2018

(30) Foreign Application Priority Data
Jun. 6, 2016 (FI) 20165467

(51) Int. Cl. A61J 1/16; G16H 20/10

(52) U.S. Cl. CPC A61J 1/165; A61J 1/22; G16H 20/10

(57) ABSTRACT

method for managing medication of a user, wherein a container for medicaments includes a storage chamber and a cap for closing the storage chamber is used, wherein the containe has a temperature sensor for measuring temperature inside the container; a humidity sensor for measuring humidity inside the container; an accelerometer sensor arranged in connection with the cap; communication means; and a first connecting part and a second connecting part arranged to attach to each other via magnetic forces. The method includes pre-setting the magnetic force between the first connecting part and the second connecting part to correspond to a given type of container; and recognising the type of container by the acceleration of the second connecting part during the attachment of the second connecting part to the first connecting part.

### TECHNICAL FIELD

The present disclosure relates generally to storage and transportation of medicines, and more specifically, to a method for managing medication of a user.

### BACKGROUND

A cold chain is a temperature-controlled supply chain. An unbroken cold chain is an uninterrupted series of storage and distribution activities which maintain a given temperature range. The cold chain is used to help extend and ensure the shelf life of products such as fresh agricultural produce, seafood, frozen food, photographic films, chemicals, and pharmaceutical drugs. Such products are referred to as ‘cool cargo’ during transport and when in transient storage. Unlike other goods or merchandise, cold chain goods are perishable and always en route towards their end use or destination, even when held temporarily in cold stores. Hence, they are commonly referred to as cargo during their entire logistics cycle. Cold chain systems protect pharmaceutical drugs and heat-labile biological preparations (e.g. sera and vaccines) from deterioration in tropical regions, which is a vital feature of immunization programs in such regions. Some medicines need to be stored at a low temperature, i.e. temperature below the normal room temperature even in colder climates.

A cooler bag may be used to carry small quantities of products (e.g. foods, pharmaceutical drugs, etc.). The cooler bag is easy to use, clean, move to other places, and it can also be reused often. The cooler bag keeps the medicines chilled or frozen (e.g., for a day) for example by using eutectic gel. However, the cooler bag can be used only for relatively short journeys to hospitals or dispensing pharmacies due to its limited range, which when exceeded, causes the cold chain to break. While transporting medicines, it is important to know how long the use “range” is, (e.g., there is need to have an indication that the drugs stored in a cold storage container can be transported for example for a maximum of 5 hours or 300 km before the medicines get too warm).

An additional challenge is that some drugs may need to be mixed, and the process of remembering whether the medicine has already been mixed or not may be tedious and error-prone, particularly for elderly patients. Users of medicines may forget the last time the drug was consumed. Further, doctors or medical staff find it difficult to keep track of how the patient takes the medicine in everyday life. Some patients adhere to medicine intake guidelines, but others do not take medicines correctly, despite instructions. They may also forget to carry their medicines while leaving for a journey, or forget to order a new batch of medicines. There are existing systems that include small fridges for storing and/or monitoring medicines, but they are not easy to transport.

Document US 2016/0120758 discloses a medicine pill box equipped with sensors to detect presence or absence status of pills and/or movement of the pill box and/or removal of a bottle cap, and a communication module. Document CN 204411275U discloses a medicament box comprising a control module comprising a sensor and a transceiver. The sensor comprises an acceleration sensor, a temperature sensor and a humidity sensor. Document WO 2015/002492 presents a medication tracker including an elastic band member configured to be detachably attached to an outside of a medicine case, a switch inside of the elastic band member to sense detachment of attachment of the elastic band member and an audio output unit. Document US 2015/0257981 discloses a monitor for monitoring consumption of pills from a container, which monitor comprises multiple sensors, an alert element and a transceiver. The sensors are configured to detect an attempt to consume a pill and to evaluate an amount of pills in the container. Document US 2016/0095797 presents a medication dispensing assembly including a canister to which a processor is coupled. A timer is coupled to the canister and the processor and the timer determines a trigger time that the medication is to be removed from the canister. The assembly further comprises an alarm and a sensor.

Therefore, in light of the foregoing discussion, there exists a need to overcome the aforementioned drawbacks in existing approaches for managing the storage and for monitoring consumption of medicines by a user.

### SUMMARY

The present disclosure provides a method for managing medication of a user, wherein a container for medicaments comprising a storage chamber and a cap for closing the storage chamber is used. The container comprises

a temperature sensor for measuring temperature inside the container;

a humidity sensor for measuring humidity inside the container;

an accelerometer sensor arranged in connection with the cap;

communication means; and

a first connecting part and a second connecting part arranged to attach to each other via magnetic forces.

The method comprises pre-setting the magnetic force between the first connecting part and the second connecting part to correspond to a given type of container and recognising the type of container by the acceleration of the second connecting part during the attachment of the second connecting part to the first connecting part.

The present disclosure also provides a container for medicine that comprises a storage chamber and a cap for closing the storage chamber. The container further comprises a temperature sensor for measuring temperature inside the container, a humidity sensor for measuring humidity inside the container, an accelerometer sensor arranged in connection with the cap, communication means, a first connecting part and a second connecting part arranged to attach to each other via magnetic forces.

The present disclosure further provides a system for monitoring medication of a user, comprising a container as disclosed above, a server and a mobile device comprising a user interface.

Embodiments of the present disclosure substantially eliminate or at least partially address the aforementioned problems in the prior art, and manage the storage and also monitor the consumption of medicines by a user.

Additional aspects, advantages, features and objects of the present disclosure are made apparent from the drawings and the detailed description of the illustrative embodiments below.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a system, in accordance with an embodiment of the present disclosure;

FIG. 2A is a functional block diagram of a server, in accordance with an embodiment of the present disclosure;

FIG. 2B is an exemplary tabular view of a server database of FIG. 2A in accordance with an embodiment of the present disclosure;

FIG. 3 is a functional block diagram of a mobile device in accordance with an embodiment of the present disclosure;

FIG. 4A is a user interface view of a temperature information processing module of FIG. 2A, in accordance with an embodiment of the present disclosure;

FIGS. 4B-4C are user interface views of a group management module of FIG. 2A, in accordance with an embodiment of the present disclosure;

FIG. 4D is a user interface view of a threshold temperature recommendation module of FIG. 2A, in accordance with an embodiment of the present disclosure;

FIG. 5A is a user interface view of a medicine type selection module of FIG. 3, in accordance with an embodiment of the present disclosure;

FIG. 5B is a user interface view of a threshold temperature information communicating module of FIG. 3, in accordance with an embodiment of the present disclosure;

FIGS. 5C-5D are user interface views of a temperature information displaying module of FIG. 3, in accordance with an embodiment of the present disclosure;

FIG. 5E is a user interface view of a historical temperature data displaying module of FIG. 3, in accordance with an embodiment of the present disclosure;

FIG. 5F is a user interface view of a device settings module of FIG. 3, in accordance with an embodiment of the present disclosure;

FIG. 5G is a user interface view of a threshold temperature recommendation obtaining module of FIG. 3, in accordance with an embodiment of the present disclosure;

FIG. 6A is a partial exploded view of a container in accordance with an embodiment of the present disclosure;

FIG. 6B is a perspective view of another type of container in accordance with an embodiment of the present disclosure;

FIG. 6C is a perspective view of the cap in accordance with an embodiment of the present disclosure;

FIG. 7 is a perspective view of an accelerometer sensor in accordance with an embodiment of the present disclosure;

FIG. 8 is an exemplary view of temperature sensitive medicines in accordance with an embodiment of the present disclosure;

FIG. 9 is a mechanical structure of a container in accordance with an embodiment of the present disclosure;

FIGS. 10A-10B are flow diagrams that illustrate a method in accordance with an embodiment of the present disclosure;

FIGS. 11A-11B are flow diagrams that illustrate a method in accordance with an embodiment of the present disclosure;

FIG. 12 is a flow diagram that illustrates a method in accordance with an embodiment of the present disclosure;

FIG. 13 is a flow diagram that illustrates a method in accordance with an embodiment of the present disclosure; and

FIG. 14 is a functional block diagram of a container, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

The present disclosure provides a method for managing medication of a user, wherein a container for medicaments comprising a storage chamber and a cap for closing the storage chamber is used. The container comprises

a temperature sensor for measuring temperature inside the container;

a humidity sensor for measuring humidity inside the container;

an accelerometer sensor arranged in connection with the cap;

communication means; and

a first connecting part and a second connecting part arranged to attach to each other via magnetic forces.

The method comprises pre-setting the magnetic force between the first connecting part and the second connecting part to correspond to a given type of container and recognising the type of container by the acceleration of the second connecting part during the attachment of the second connecting part to the first connecting part.

The present disclosure provides a container for medicine that comprises a storage chamber and a cap for closing the storage chamber. The container further comprises a temperature sensor for measuring temperature inside the container, a humidity sensor for measuring humidity inside the container, an accelerometer sensor arranged in connection with the cap, communication means, a first connecting part and a second connecting part arranged to attach to each other via magnetic forces.

The storage chamber thus comprises a place to store medicines. For example, the storage chamber stores an insulin pen or other temperature sensitive medicine. The storage chamber may further comprise a place to store a cold and/or a hot gel if needed. The storage chamber may also be simply well isolated and hence not need any cold or hot gel. The present container may also be used for monitoring and/or managing medication of a user, in which case the temperature of the storage is not relevant, i.e. no hot or cold gels (or similar products) are needed. The storage chamber of the container may also comprise place for silica gel or similar product that absorbs excess moisture.

The cap is arranged to close the storage chamber. The cap can be made of one part or it may comprise several part, such as two, three or four parts. Ideally, the cap is such that it can be easily opened and closed, while also being secure such that the container can be transported in any position.

The container further comprises a temperature sensor, a humidity sensor and an accelerometer sensor. The temperature sensor and the humidity sensor are integrated with at least one of the cap, or the container. The accelerometer sensor is arranged in connection with the cap, i.e. it may be integrated in the cap. These sensors can be any suitable sensors known per se, as long as they are small enough for this use. The sensors are ideally communicatively coupled to the communication means and are configured to communicate their measurement data to the communication means.

The accelerometer sensor senses an acceleration of the cap to determine opening and closing of the cap. This information can be used for example for monitoring the medication (with the assumption that when the cap is opened, some medication is taken out from the container), but also for monitoring temperature of the container, i.e. temperature inside the container, in the storage chamber. Vice versa, opening and closing of the cap may also be determined by monitoring changes in temperature of the container.

Furthermore, in case the position of the accelerometer sensor relative to the cap is known, the position of the container can be determined (for example whether its top is up or down). This information can also be used to determine whether the cap is being attached to the container body or detached from the container body.

The container further comprises communication means that may be configured to communicate signals to at least one of a server or a mobile device. The communication may be carried out for example using a radio link, a wireless communication, a wired communication, a wireless local area network (WLAN), or a local area network (LAN).

The temperature sensor, the humidity sensor and the communication means can be arranged into the storage chamber or the cap as required, while bearing in mind their function.

The container also comprises a first connecting part and a second connecting part. The first connecting part and the second connecting part are arranged to attach to each other via magnetic forces. According to an embodiment, there are several different connecting parts, or mainly their magnetic forces required for closing can be different. As such combinations are entered to a database and the acceleration sensor provides acceleration data when the cap is closed and/or opened, the specific acceleration detected can be coupled with the information in the database and thus the type of the container can be detected and identified. For example, the magnetic force between the two connecting parts can be relative to the size of the container, i.e. a smaller container is equipped with connecting parts using a lower magnetic force, and a larger container equipped with connecting parts using a higher magnetic force. As the magnetic force can be indirectly determined with the acceleration sensors, the size of the container can be determined when comparing the magnetic force to a database of known pairs of connecting parts. In other words, the first connecting part and the second connecting part form a reproducible and measurable response that can be measured using the accelerometer sensor. As another example, magnetic forces can be used to identify same size (and/or form factor) containers from each other (for example container A from container B). The magnetic forces between two connecting parts could be different for the container A in comparison to magnetic forces between two connecting parts for the container B. The magnetic force causes different accelerations for the cap when the container is closed/opened thus enabling to identify container A closing/opening from container B closing/opening.

The container also comprises other parts required for its use according to the present description, such as an energy source. The energy source can be for example a battery, or the container may comprise means for storing kinetic energy.

According to an embodiment, the first connecting part comprises magnetic material and the second connecting part comprises magnetic material or ferromagnetic material. According to another embodiment, the first connecting part is arranged in the storage chamber and the second connecting part is arranged in the cap. According yet another embodiment, the first connecting part is arranged in a first part of the cap and the second connecting part is arranged in a second part of the cap, which second part of the cap is arranged in the first part of the cap.

Indeed, the first connecting part can be arranged in the storage chamber and the second connecting part in the cap. In this embodiment, opening and closing the container can be measured in various manners, for example by measuring the acceleration of the cap itself or by measuring the acceleration of the cap with respect to the storage chamber (due to the magnetic forces). When both connecting parts are arranged in the cap, the measurement of the magnetic force is done typically only once, when the cap is manufactured or assembled. Thereafter, the connecting parts are not necessarily taken apart again.

However, according a yet further embodiment, the cap comprises a removable component, which component comprises the accelerometer sensor and the second connecting part. According to another embodiment, the removable component comprises the temperature sensor, the humidity sensor, the accelerometer sensor, the communication means and the second connecting part. In these embodiments, and especially with the second embodiment, the removable component can be easily replaced when for example the battery is worn out.

According to yet another embodiment, the user can indicate that she/he has taken the medicine for example by tapping on the cover in a pre-defined manner (for example two or three consecutive taps). This tapping is recorded by the accelerometer sensor and communicated further.

The container can be made up of any suitable material, such as polyolefins, polyvinyl chloride, ethylene-vinyl acetate copolymer, or a metallic material such as aluminum or iron. The storage chamber and the cap can be made of a same material or of different materials.

The container can be used as a transportation bag/a storage unit of medicines. The present container can thus be used to monitor whether the appropriate temperature is maintained inside the container to keep the medicines safe during transportation and/or storage.

The present disclosure also relates to a system for monitoring medication of a user. In this case, the system further comprises a server and a mobile device comprising a user interface. The server may be for example a desktop, a mobile phone, a smart phone, a tablet, a personal computer, or an electronic notebook, etc. A mobile device can thus function also as a server. The mobile device can be a mobile phone, a smart phone, a table, an electronic notebook, a smart watch or similar.

The server may comprise for example a threshold temperature information obtaining module, a container temperature information obtaining module, a temperature information processing module, an alarm communicating module, a group management module, a threshold temperature recommendation module, and a server database. The threshold temperature information obtaining module is preferably configured to obtain threshold temperature limit of the container for a selected type of medicine from the mobile device, and for example store in on the database. The container temperature information obtaining module is preferably configured to obtain temperature inside the container from the sensor unit of the container, i.e. the temperature sensor senses the temperature inside the container and communicates the temperature information to the server.

The temperature information processing module is preferably configured to determine whether the temperature inside the container exceeds the threshold temperature limit (e.g., a soft temperature limit, or a hard temperature limit). When the temperature inside the container exceeds the threshold temperature limit, the alarm communicating module communicates a sound or visual indication on the mobile device to pay attention to storage conditions of the container and inside the container. For example, the database can contain four different temperature limits: a soft lower temperature limit, a hard lower temperature limit, a soft higher temperature limit and a hard higher temperature limit. The soft temperature limits are limits that raise an alarm that a change in the storage conditions is necessary for keeping the medicine usable. The hard temperature limits are limits below or above which the medicine is already unusable, i.e. deteriorated, and must be disregarded. The alarms for these limits are preferably set to be different, so as to clearly indicate to the user which temperature limit is exceeded.

The group management module is preferably configured to provide options to a user (e.g. a group admin) of the server to provide for example a read-only access to a third party (e.g. other members). Further, the group management module is preferably configured to provide options to the user to review a history of measurement data of containers of members of the user's group. The threshold temperature recommendation module is preferably configured to recommend a new threshold temperature limit for the container to the mobile device. The threshold temperature recommendation module may provide information of the container and instruction to the user to the mobile device. The server database may store container information (e.g., temperature and/or humidity inside the container, battery status of the container, or amount of medicines remaining inside the container) received from the sensor unit of the container and/or determined based on the various measurement data (acceleration data, temperature data).

The mobile device may include for example a device database, a medicine type selection module, a threshold temperature information communicating module, a temperature information displaying module, an alarm receiving module, a historical temperature data displaying module, a threshold temperature recommendation obtaining module and a device settings module. The device database may store for example historical temperature information of the container, the new threshold temperature limit of the container received from the server and/or the threshold temperature limit of the container. The medicine type selection module may provide for example an option to a device user to select a type of medicine available in the container. The threshold temperature information communicating module may communicate for example the threshold temperature information of the container for the selected type of medicine to the server. The temperature information displaying module may obtain for example the temperature of the container from the server, and display the temperature inside the container in the mobile device. The alarm receiving module may receive for example the sound or the visual indication to pay attention to the storage conditions when the temperature of the container exceeds the threshold temperature limit. The alarm receiving module may further receive the alarm (for example sound, vibration or visual indication).

The historical temperature data displaying module may obtain for example historical temperature data of the container from the server and display the historical temperature data. The threshold temperature recommendation obtaining module may obtain for example the recommendations on at least one of a new threshold temperature limit of the container or information and instructions of the container from the server. The threshold temperature recommendation obtaining module may for example display the visual indication to replace the battery when the battery level of the container is low or empty. The device settings module may display for example at least one of a battery level of the container, information of a group (e.g. an admin group), warning temperature, type of medicines and temperature threshold limits or a disconnect option, etc.

The system may also be configured to take into account the temperature and/or humidity history inside the container, and to calculate or estimate its effect on the available storage time of the medicine. For example, although a given medicine may remain usable at a given temperature, the conditions may be such that they shorten the storage time of the medicine from that originally indicated by the medicine manufacturer.

In another aspect, a method for managing medication of a user is provided, which method is carried out using the container as described above. The method comprises pre-setting the magnetic force between the first connecting part and the second connecting part to correspond to a given type of container, and recognising the type of container by the acceleration of the second connecting part during the attachment of the second connecting part to the first connecting part. The method is thus used as explained above in connection with the description of the connecting parts.

In an embodiment, the method further comprises recognising opening and closing of the container by the acceleration of the cap, calculating the amount of remaining medicine in the container based on the number of openings of the container, communicating the amount of remaining medicine to the user, communicating the temperature and/or humidity inside the container to the user, and communicating an indication to the user when the temperature and/or the humidity is over and/or under a pre-defined first threshold. It is of course also possible that the communication of the various information listed above, or some of them, is done to different users. For example, the information can be sent to an administrator, or to the user and an administrator, or to a relative or care taker of the user. For example, it is also possible that when the amount of medicine in the container falls below a pre-defined limit, an automatic order for medicine is sent to a pharmacy or the like. Thus, according to another embodiment, the method further comprises communicating an order for medicine to a server.

According to one embodiment, the indication to the user comprises a pre-warning that within a pre-defined time, the temperature and/or the humidity are over and/or under the storage temperature and/or the humidity of the medicine. This embodiment has also been discussed in more detail above, i.e. the pre-warning is related to a so-called soft temperature limit.

According to another embodiment, the indication to the user comprises a warning that indicates that the medicine is no longer usable. The cap may comprise an indicator (e.g. a LED) that visually indicates error conditions. For example, when the temperature of the container exceeds the hard temperature limit, the LED indicator is lit.

According to another embodiment, the method further comprises communicating the amount of remaining medicine, and/or communicating the temperature and/or the humidity inside the container to the server, and the server providing indications to the user. The container may thus communicate either directly with the user (for example a mobile device of the user, associated with the container via a suitable app) or via a server.

The measurement data collected by the present container (for example temperature, humidity, acceleration, GPS-location, atmospheric pressure) may be combined with measurement data obtained from other sources, such as databases. Such databases may contain for example recorded temperature data from anywhere in the earth, amount of traffic, transportation time tables etc. These data combined will then form a database that functions as a training data set for the container. This training data set is used to train a Recurrent Neural Network (RNN). The most notable feature of a RNN is the memory effect, whereby the algorithm learns how to recognise recurring patterns. As an end result, the trained RNN can for example give an estimate of how long the medications stored in the container remain usable or when a new set of medication should be ordered. A trained RNN is a significantly more accurate way of predicting than traditional methods based on regression or statistics. The embodiments of the present description can take the form of an entirely hardware embodiment, an entirely software embodiment or an embodiment including both hardware and software elements. The embodiments that are implemented in software include but are not limited to firmware, resident software, microcode, etc. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate or transport the program for use by or in connection with the instruction execution system, apparatus or device.

The medium can be an electronic, magnetic, optical, electromagnetic, infrared or semiconductor system (or apparatus or device) or a propagation medium. Examples of a computer-readable medium include a semiconductor or solid-state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD.

A data processing system suitable for storing and/or executing a program code may include at least one processor coupled directly or indirectly to memory elements through a system bus. The memory elements can include local memory employed during actual execution of the program code, bulk storage and cache memories which provide temporary storage of at least some program code in order to reduce the number of times the code must be retrieved from the bulk storage during execution.

Input/output (I/O) devices (including but not limited to keyboards, displays, pointing devices, remote controls etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

### DETAILED DESCRIPTION OF THE DRAWINGS

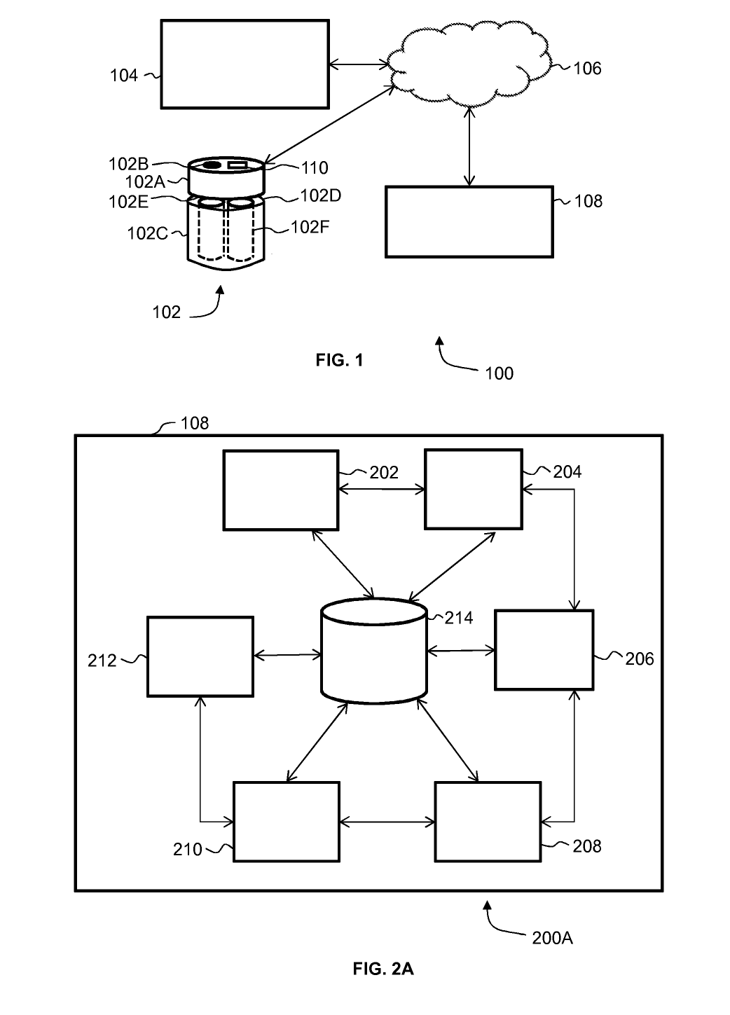

FIG. 1 is a schematic illustration of a system 100, in accordance with an embodiment of the present disclosure. The system 100 includes a container 102, a mobile device 104, a network 106 and a server 108. The container 102 further includes a cap 102A, a sensor unit 102B, a container body 102C, a first connecting part 102D, a second connecting part 102E and a communication unit 110. The container body 102C includes a storage chamber 102F that can store a variety of medicines. The cap 102A is arranged to close the container body 102C. The sensor unit 102B and the communication unit 110 may be arranged in connection with the cap 102A. The sensor unit 102B includes a temperature sensor for measuring temperature inside the container 102, a humidity sensor for measuring humidity inside the container 102 and an acceleration sensor that is arranged in connection with the cap 102A. The sensor unit 102B is communicatively coupled to the communication unit 110 to communicate measurement data. The communication unit 110 is communicating with the server 108 directly over the network 106 or via the mobile device 104 in order to manage and monitor the medicines that are stored in the storage chamber 102F of the container 102. The communication unit 110 communicates container information of the container 102 to at least one of the mobile device 104 or the server 108. The first connecting part 102D may be arranged in connection with the container body 102C. The second connecting part 102E may be arranged in connection with the cap 102A. The first connecting part 102D, and the second connecting part 102E are arranged to attach to each other using magnetic forces. The first connecting part 102D and the second connecting part 102E may include a magnetic material or a ferromagnetic material. The server 108 receives the container information (e.g., a temperature and/or humidity inside the container 102, a battery status of the container 102, or an amount of the medicines available inside the container 102) from the communication unit 110. The server 108 processes the container information, and transmits it to the mobile device 104.

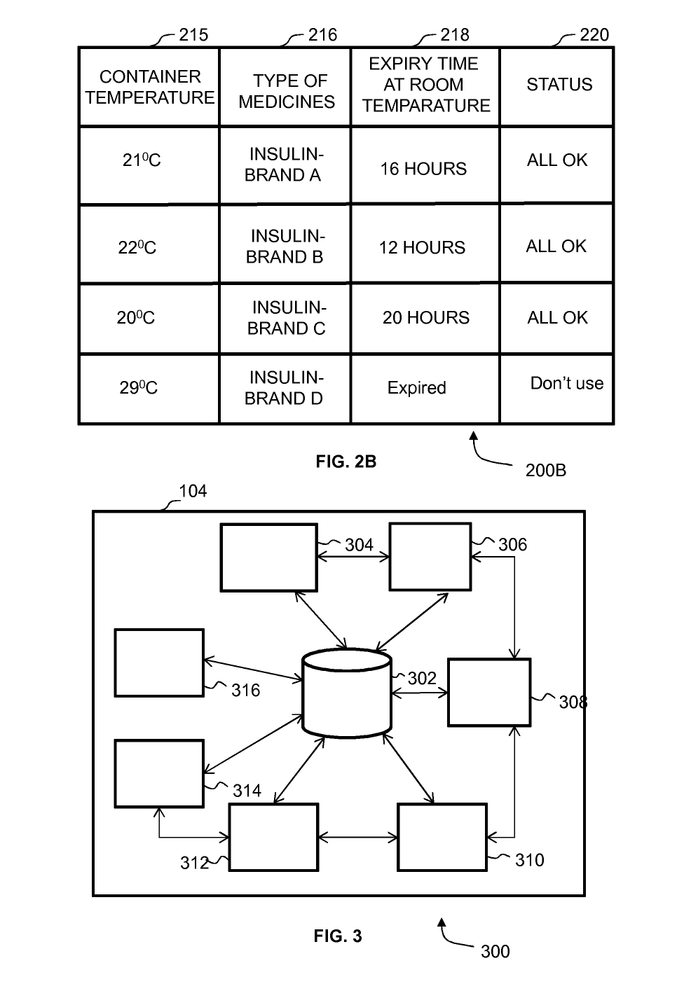

Referring to FIG. 2A, illustrated is a functional block diagram 200A of a server 108 (for example of the server of FIG. 1), in accordance with an embodiment of the present disclosure. The functional block diagram 200A of the server 108 includes a threshold temperature information obtaining module 202, a container temperature information obtaining module 204, a temperature information processing module 206, an alarm communicating module 208, a group management module 210, a threshold temperature recommendation module 212, and a server database 214. These modules function as has been described above.

Referring to FIG. 2B, illustrated is an exemplary tabular view 200B of the server database 214 of FIG. 2A that illustrates storage of temperature information of the container 102, in accordance with an embodiment of the present disclosure. The exemplary tabular view 200B of the server database 214 includes a container temperature field 215, a type of medicines field 216, an expiry time field 218 and a status field 220. The container temperature field 215 stores a temperature inside the container 102. The type of medicines field 216 stores a type of medicine available in the container 102. The expiry time field 218 stores a time of expiry of the medicine based on the temperature inside the container 102. The status field 220 stores a status of the medicine, i.e. whether it is usable or not based on the temperature inside the container 102 and the type of medicine stored inside the container 102.

Referring to FIG. 3, illustrated is a functional block diagram 300 of the mobile device 104, in accordance with an embodiment of the present disclosure. The functional block diagram 300 of the mobile device 104 includes a device database 302, a medicine type selection module 304, a threshold temperature information communicating module 306, a temperature information displaying module 308, an alarm receiving module 310, a historical temperature data displaying module 312, a threshold temperature recommendation obtaining module 314, and a device settings module 316, as discussed above.

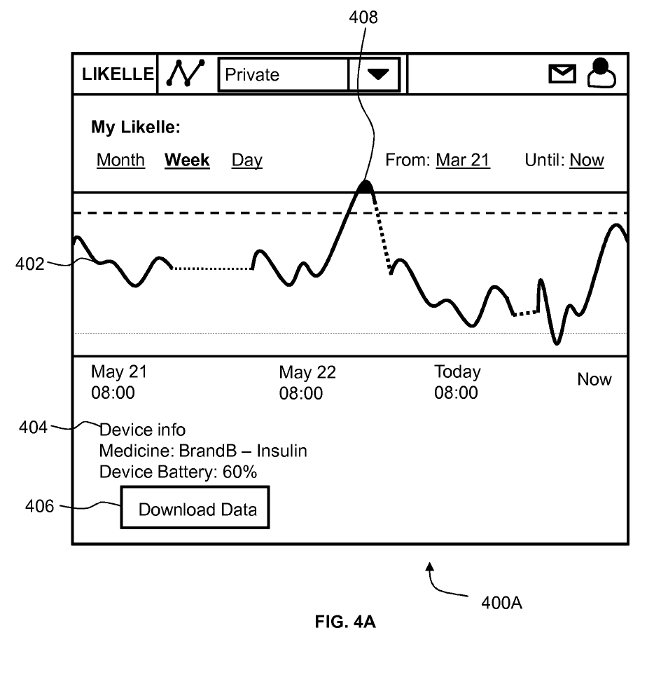

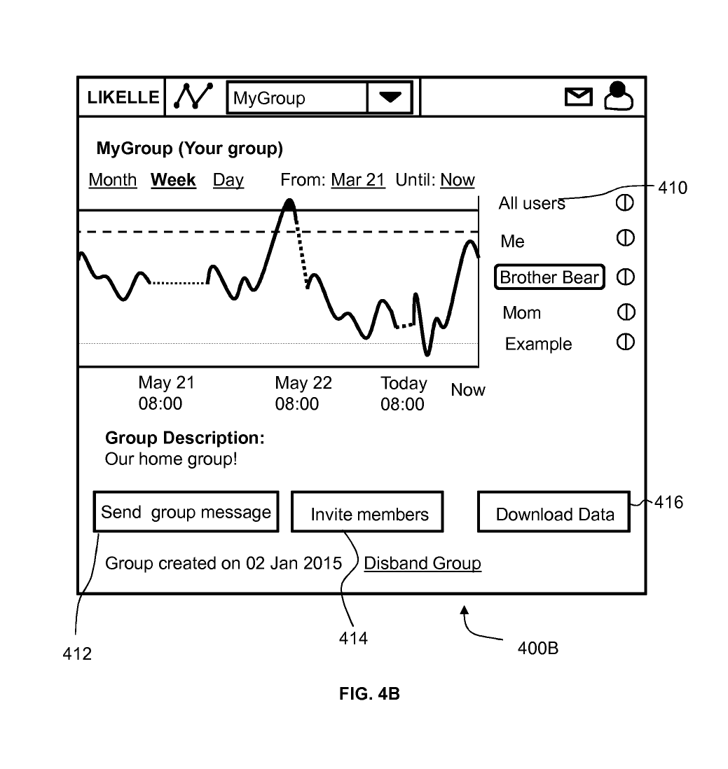

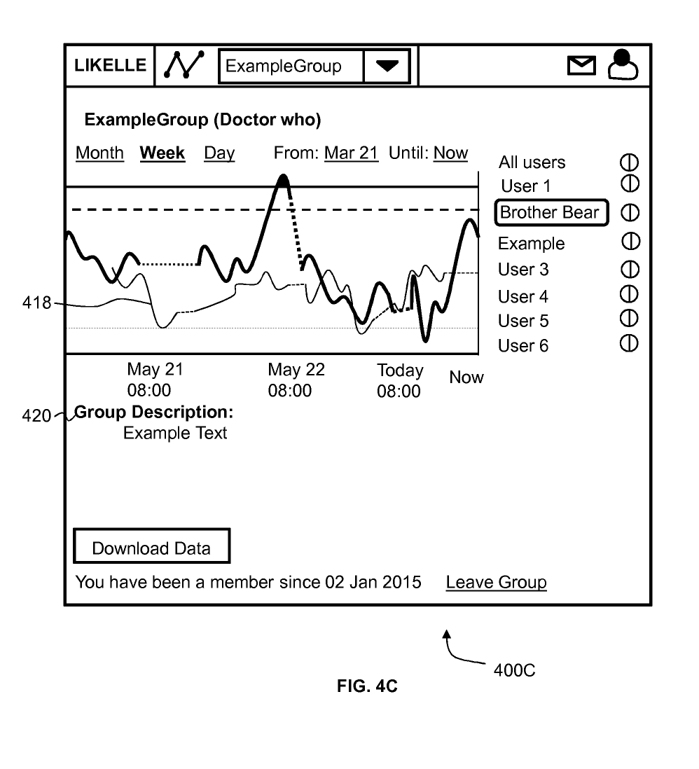

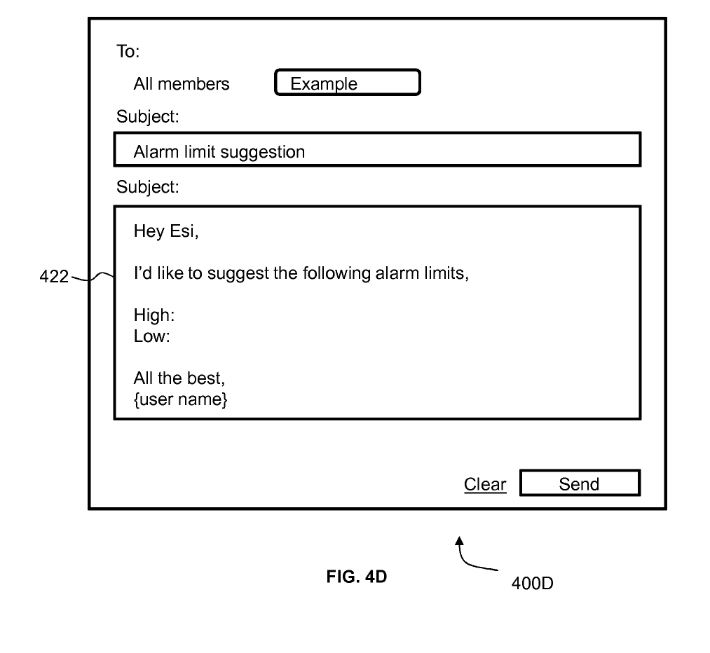

Referring to FIG. 4A, illustrated is a user interface view 400A of the temperature information processing module 206 of FIG. 2A, in accordance with an embodiment of the present disclosure. The temperature information processing module 206 processes the temperature inside the container 102 and displays historical temperature data 402 of the container 102. The user interface view 400A of the temperature information processing module 206 further displays the container information 404 that includes a type of medicine available in the container 102 and a battery level of the container 102. The user interface view 400A of the temperature information processing module 206 includes a download data field 406 that provides options to download the container information 404 as a separate data file. The user interface view 400A of the temperature information processing module 206 includes an indicator field 408 that provides an indication, when the temperature inside the container 102 exceeds the threshold temperature limit. The alarm communicating module 208 of the server 108 communicates an alarm (e.g., a sound, a vibration, or a visual message) to the mobile device 104 to pay the attention to storage conditions of the container 102, when required.

Referring to FIGS. 4B-4C, illustrated are user interface views of the group management module 210 of FIG. 2A, in accordance with an embodiment of the present disclosure. The user interface view 400B of the group management module 210 includes an all users field 410, a group message field 412, an invite member field 414, and a download data field 416. The all users field 410 displays a list of members belonging to a group. The user interface view 400B of the group management module 210 provides options to the user (e.g. the group administrator) to provide a read only access to a third party user. The user interface view 400B further provides options to the user of the server 108 to review a history of the measurement of the members in a group. The group message field 412 provides options to the user to send a message to all the members of a group. The invite member field 414 provides options to the user to invite new members to a group. The download data field 416 provides options to download group information as a separate file.

The user interface view 400C includes a normalized history data field 418 and a group description field 420. The normalized history data field 418 displays historical temperature data normalized to the threshold temperature limits of the container 102. The group description field 420 displays a description of the user group based on at least one of users of a medicine type, geographical locations or any other common characteristic.

Referring to FIG. 4D, illustrated is a user interface view 400D of the threshold temperature recommendation module 212 of FIG. 2A, in accordance with an embodiment of the present disclosure. The threshold temperature recommending module 212 automatically communicates a recommendation message 422 to the mobile device 104. The recommendation message 422 includes at least one of the device information, instructions or a new threshold limit.

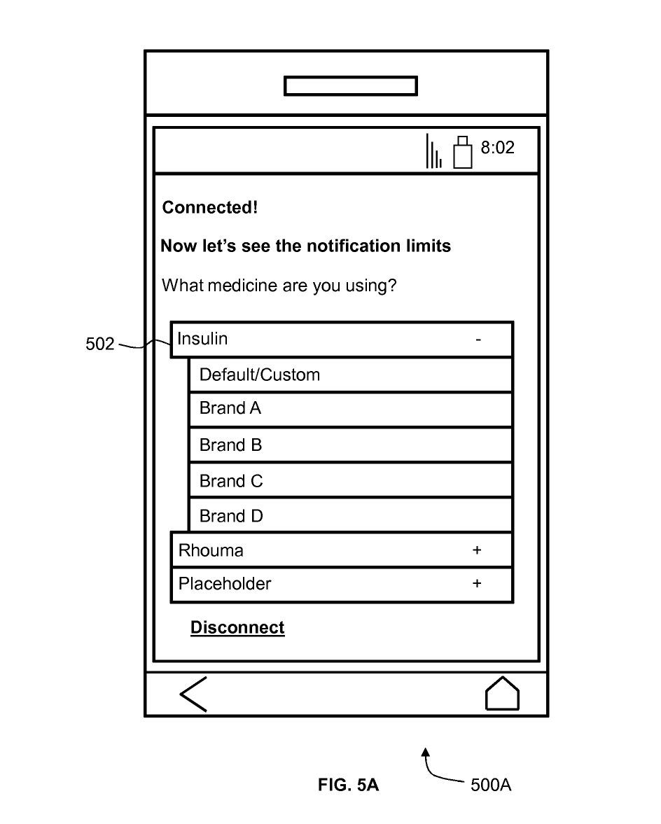

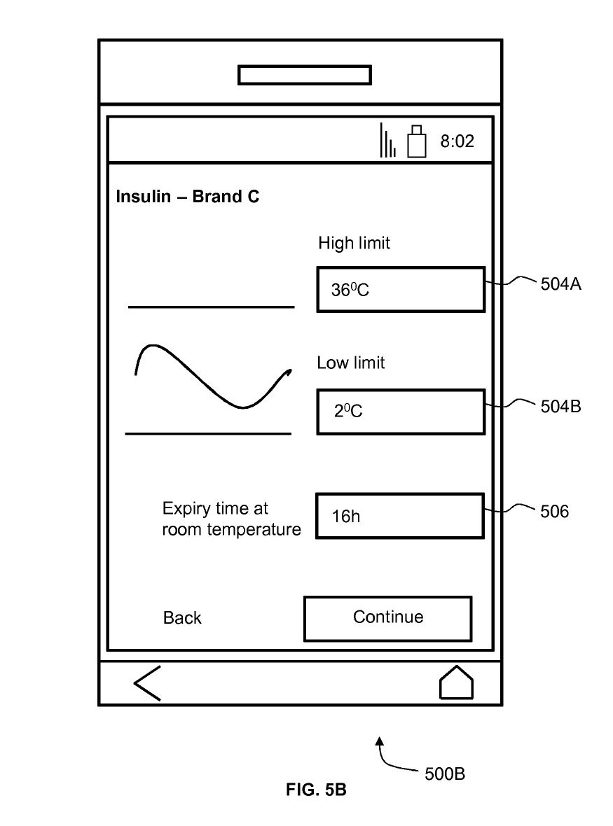

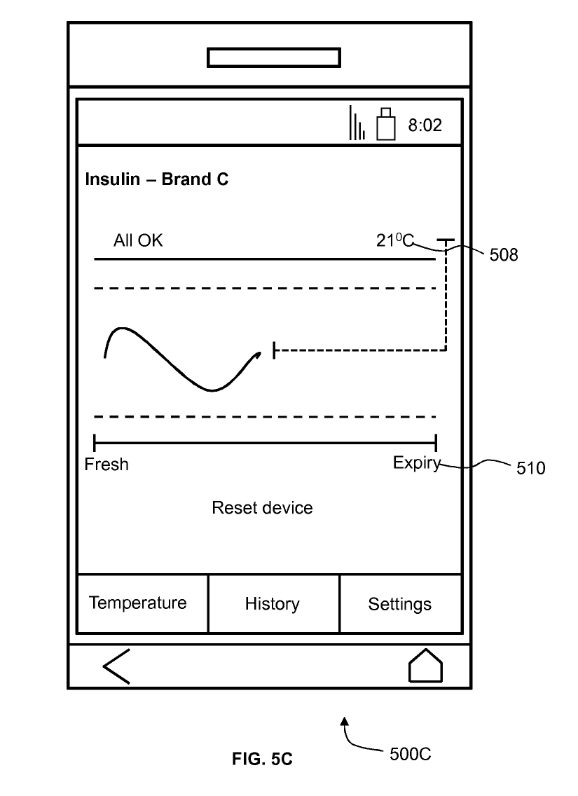

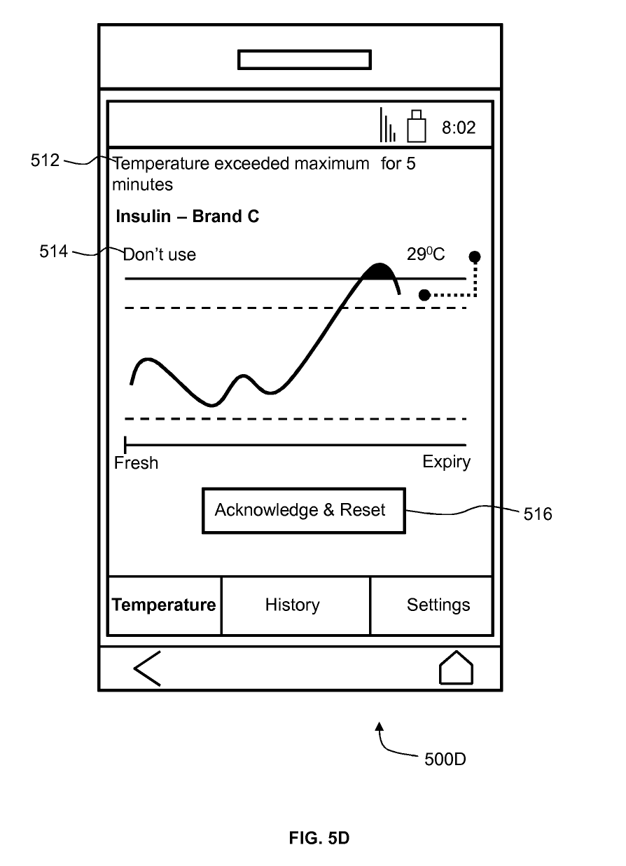

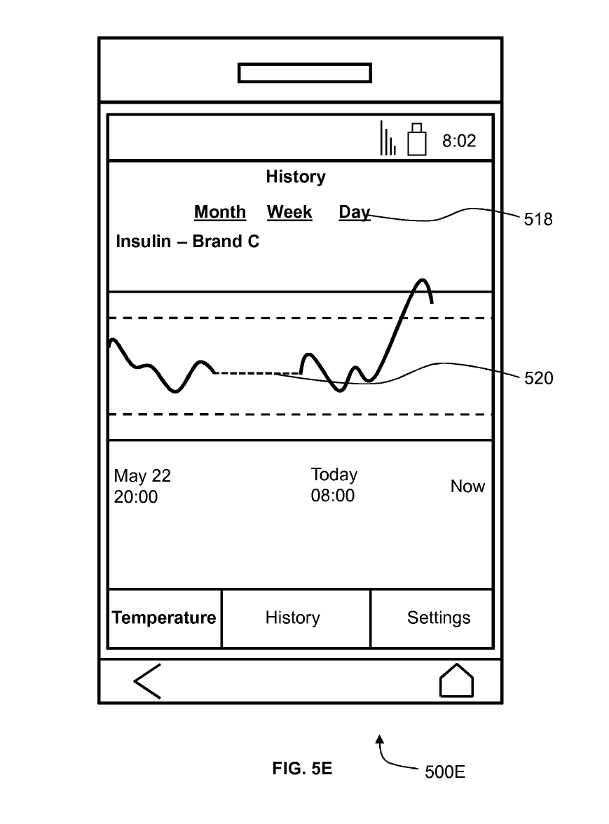

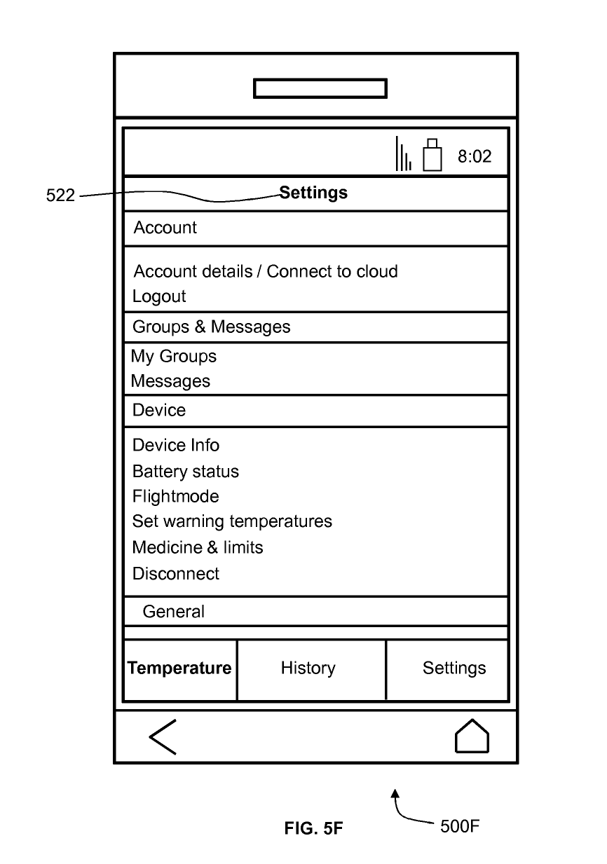

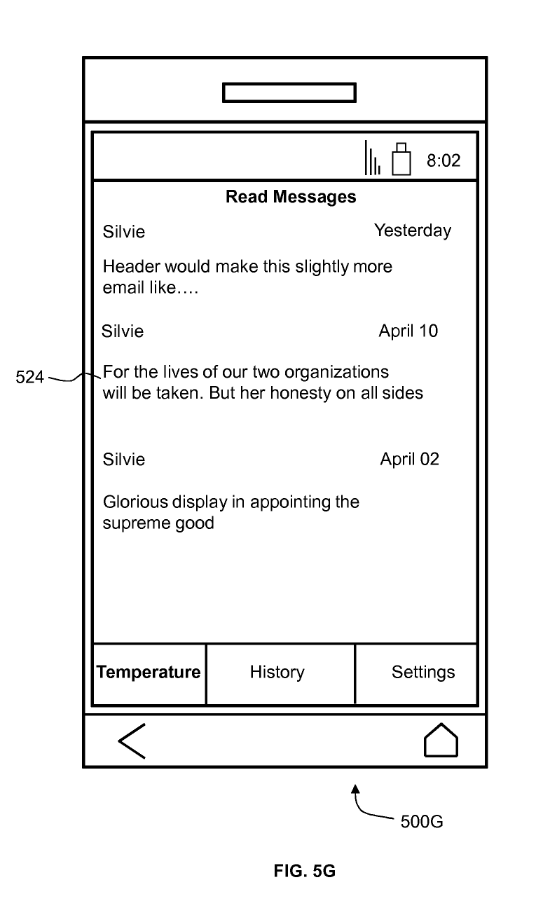

Referring to FIG. 5A, illustrated is an user interface view 500A of the medicine type selection module 302 of FIG. 3, in accordance with an embodiment of the present disclosure. The users interface view 500A provides an option to the device user to select a type of the medicines 502 that the device user uses. The medicine type selection module 302 provides options to the device user to select whether the device user uses medicines that are stored at room temperature or in refrigerated conditions.

Referring to FIG. 5B, illustrated is an user interface view 500B of the threshold temperature information communicating module 306 of FIG. 3, in accordance with an embodiment of the present disclosure. The user interface view 500B includes temperature limits field 504A-B and an expiry time of the medicines field 506. The temperature limits field 504A-B provides an option to the device user to pre-set the threshold temperature limits (i.e. the high temperature limit and the low temperature limit for a medicine). The expiry time of the medicines field 506 provides an option to the device user to set a length of the time that the medicine remains usable in the room temperature.

Referring to FIGS. 5C-5D, illustrated are user interface views of the temperature information displaying module 308 of FIG. 3, in accordance with an embodiment of the present disclosure. The temperature information displaying module 308 obtains the temperature of the container 102 from the server 108 and displays the temperature inside the container 102 in the mobile device 104. For example, the user interface view 500C of the temperature information displaying module 308 displays a current temperature 508 (e.g. 21° C.) of the container 102, and an estimation of how long the medicine is useful in the current temperature 510 based on a historical temperature data slope.

The user interface view 500D of the temperature information displaying module 308 displays the temperature inside the container 102. The user interface view 500D of the temperature information displaying module 308 displays a current temperature (e.g. 29° C.) of the container 102, and displays for how long the temperature exceeded hard temperature limit (e.g. temperature exceeded hard temperature limit for 5 minutes) at 512. The user interface view 500D of the temperature information displaying module 308 displays a “Don't use”-message 514 in the mobile device 104. The user interface view 500D includes an acknowledgement and reset field 516 that provides an option to the device user to acknowledge to the server 108 and/or reset the mobile device 104.

Referring to FIG. 5E, illustrated is an user interface view 500E of the historical temperature data displaying module 312 of FIG. 3, in accordance with an embodiment of the present disclosure. The user interface view 500E of the historical temperature data displaying module 312 displays history of temperature data of the container 102. When the connection between the mobile device 104, and the server 108 fails, and the history of temperature is not available, the user interface view 500E displays this visually (shown as 520, in this embodiment a straight line, possibly rendered in another colour) to the device user. The user interface view 500E provides an option 518 to the device user to view the historical temperature data on monthly basis, weekly basis or daily basis.

Referring to FIG. 5F, illustrated is a user interface view 500F of the device settings module 316 of FIG. 3, in accordance with an embodiment of the present disclosure. The user interface view 500F provides options 522 to include at least one of information of the container 102 (e.g. the container information, the battery status, a Flight mode option, warning temperature settings, medicines and limits, disconnect option, and general options), account details or group and messages details.

Referring to FIG. 5G, illustrated is a user interface view 500G of the threshold temperature recommendation obtaining module 314 of FIG. 3, in accordance with an embodiment of the present disclosure. The user interface view 500G of the threshold temperature recommendation obtaining module 314 obtains automated recommendations from the server 108 and displays them as messages in the mobile device 104. For example, the user interface view 500G of the threshold temperature recommendation obtaining module 314 displays a “pay attention to the temperature”-message when the temperature of the container 102 decreases below or raises above the soft temperature limit. In another example, the user interface view 500G of the threshold temperature recommendation obtaining module 314 displays a “replace the battery”-message when the battery level of the container 102 is low.

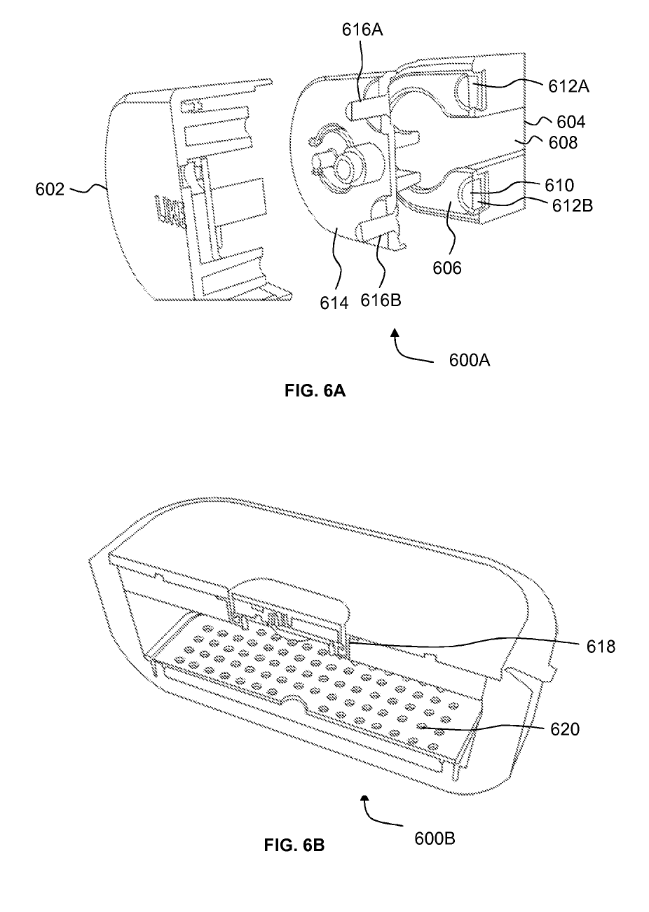

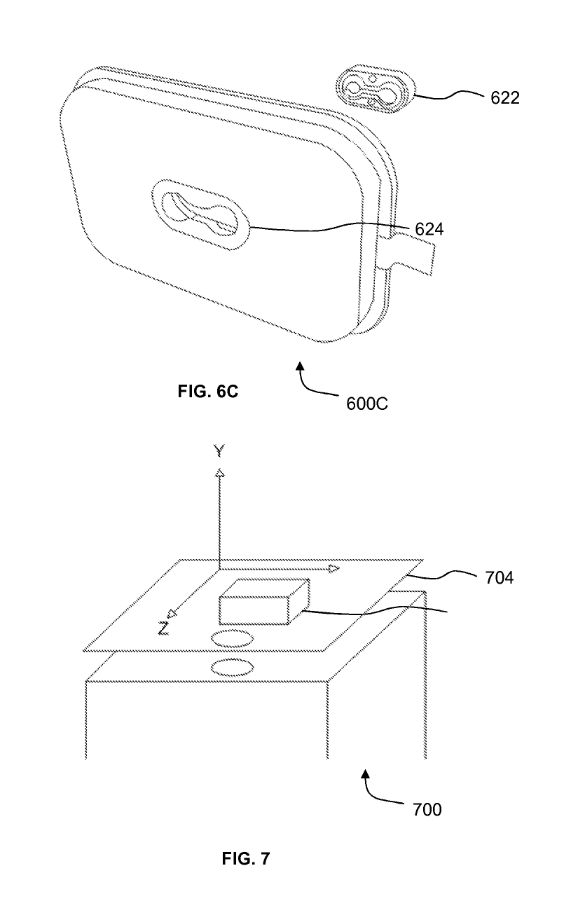

Referring to FIG. 6A, illustrated is a partial exploded view 600A of a container, in accordance with an embodiment of the present disclosure. The partial exploded view 600A includes a cap 602 and a container body 604. The container body 604 includes a first connecting part 606 and a storage chamber 608. The first connecting part 606 includes a counterpart 610, and one or more magnets 612A-B. The cap 602 includes a second connecting part 614. The second connecting part 614 includes one or more magnetic screws 616A-B. The counterpart 610 is arranged in connection with the container body 604. The counterpart 610 includes locations for the one or more magnets 612A-B. The storage chamber 608 is adapted to the container body 604 to store the medicines.

Referring to FIG. 6B, illustrated is a perspective view 600B of another type of container, in accordance with an embodiment of the present disclosure. The perspective view 600B includes a cap 618 and a storage chamber 620. The storage chamber 620 stores a variety of medicines. The cap 618 includes a counter element section that allows the cap 618 to be connected to various frames.

Referring to FIG. 6C, illustrated is a perspective view 600C of cap of FIG. 6B. The cap comprises an opening 624, into which the element 622 can be easily fitted. The element 622 comprises the sensors, the communication means and the connecting parts. In this embodiment, the element 622 is replaceable for example when its battery is worn out.

Referring to FIG. 7, illustrated is a perspective view 700 of an accelerometer sensor 702 to identify an opening position of the cap 704 of the container (as in FIG. 1), in accordance with an embodiment of the present disclosure. The perspective view 700 shows identification of opening and closing direction of the cap 704 using the accelerometer sensor 702. The identification of the opening and closing direction of the cap 704 is identified by combining the information obtained from the accelerometer sensor 702 and temperature change information.

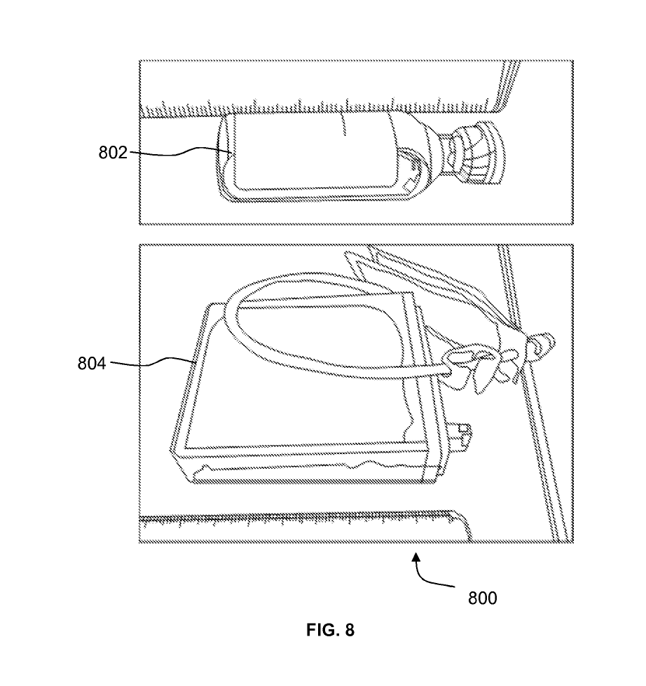

Referring to FIG. 8, illustrated is an exemplary view 800 of temperature sensitive medicines/drugs that can be packaged in various forms in the container 102, in accordance with an embodiment of the present disclosure. The exemplary view 800 shows different forms of packaging that are typically used for the temperature sensitive medicines. For example, the medicines may be packaged in an ampoule 802 or in a cylindrical cartridge 804.

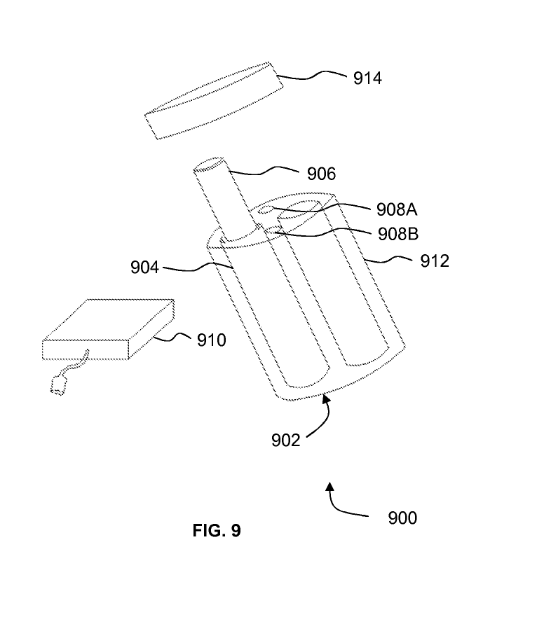

Referring to FIG. 9, illustrated is a mechanical structure 900 of a container 902 in accordance with an embodiment of the present disclosure. The mechanical structure 900 of the container 902 includes a storage chamber 904 that stores a cylindrical shaped medicine 906, one or more specific magnets 908A-B, and a rectangular shaped medicine 910. The mechanical structure 900 shows that the cylindrical shaped medicine 906 is inserted into the storage chamber 904 of a container body 912. The rectangular shaped medicine 910 is not inserted into the storage chamber 904 of the container body 912 because of the cylinder shaped container. The one or more specific magnets 908A-B are used to identify the container body 912. In an embodiment, different size container bodies include different size magnets (or magnets with different magnetic forces) that cause differentiable accelerations to a cap 914 when closing or opening the container. In another embodiment, same size container bodies can comprise different size magnets in respect to each others to cause differentiable accelerations to the cap 914 when closed and/or opened.

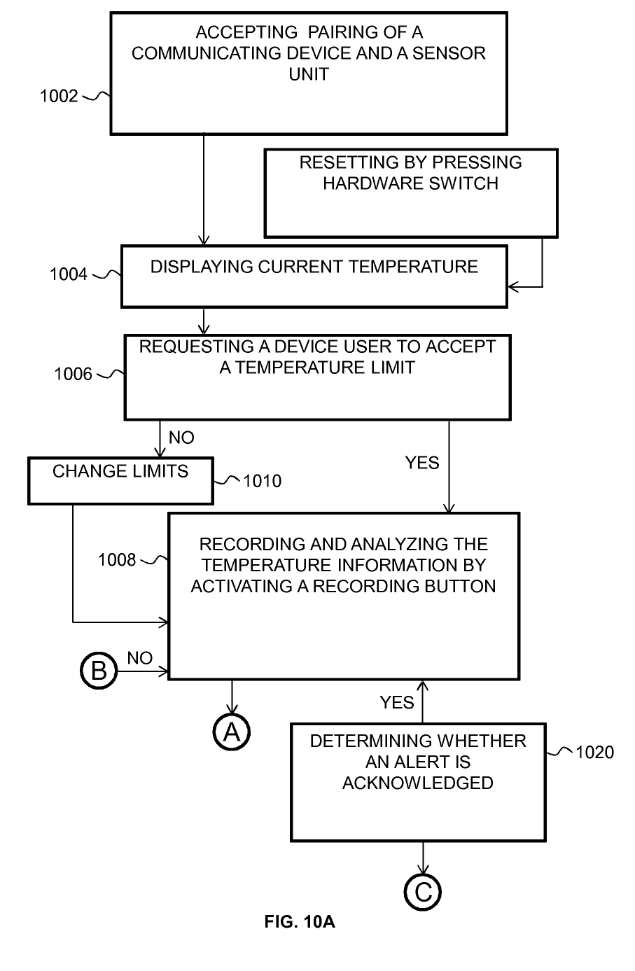

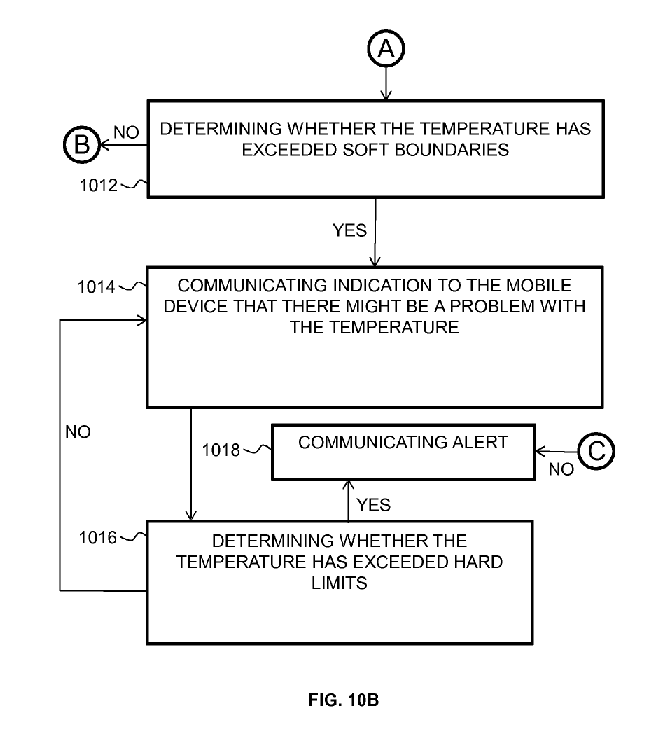

FIGS. 10A-10B are flow diagrams that illustrate a method for determining temperature inside the container 102 using the server 108, in accordance with an embodiment of the present disclosure. At step 1002, a pairing of a mobile device 104 and a sensor unit 102B is accepted. At step 1004, a current temperature inside the container 102 is displayed. In an embodiment, a hardware switch is pressed to reset the mobile device 104 to display the current temperature inside the container 102. At step 1006, a device user interacts with the mobile device 104 to accept a temperature limit. At step 1008, temperature information is recorded and analyzed by activating a recording button when the temperature limit is accepted. At step 1010, the temperature limits are changed, recorded and analyzed when the temperature limit is not accepted. At step 1012, it determines whether soft temperature limits exceed the temperature limit. If the soft temperature limits exceed the temperature limit, an indication is communicated to the mobile device 104 that there may be a problem with the temperature at step 1014. If the soft temperature limits do not exceed the temperature limit, the process starts again from the step 1008. At step 1016, it determines whether hard temperature limits exceed the temperature limit. If the hard limits exceed the temperature limit, an alert is communicated to the mobile device 104 at step 1018. If the hard temperature limits do not exceed the temperature limit, the process again starts from the step 1014. At step 1020, it determines whether the alert is acknowledged by the device user. If the alert is acknowledged by the device user, the temperature information is recorded and analyzed by activating the recording button. If the alert is not acknowledged, the alert is communicated to the mobile device 104.

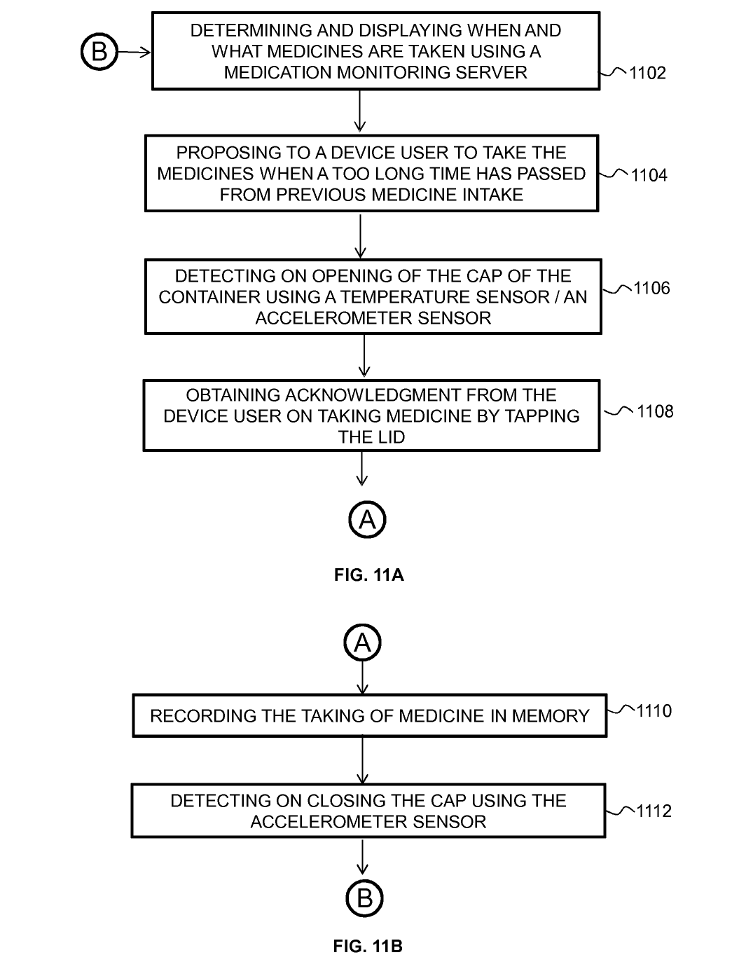

FIGS. 11A-11B are flow diagrams that illustrate a method for determining when and what medicines are taken using the server 108, in accordance with an embodiment of the present disclosure. At step 1102, usage of the medicines (e.g. when and what medicines are taken) is determined and displayed using the server 108. At step 1104, a device user is reminded to take the medicines when a too long time has passed since previous intake of the medicine. At step 1106, an opening of the cap 102A of the container 102 is detected using a temperature sensor and/or an accelerometer sensor 702. At step 1108, an acknowledgement is obtained from the device user on taking the medicine by tapping the cap 102A. At step 1110, the taken medicine is recorded in a memory. At step 1112, closing the cap 102A is detected using the accelerometer sensor 702 and the process is recycled.

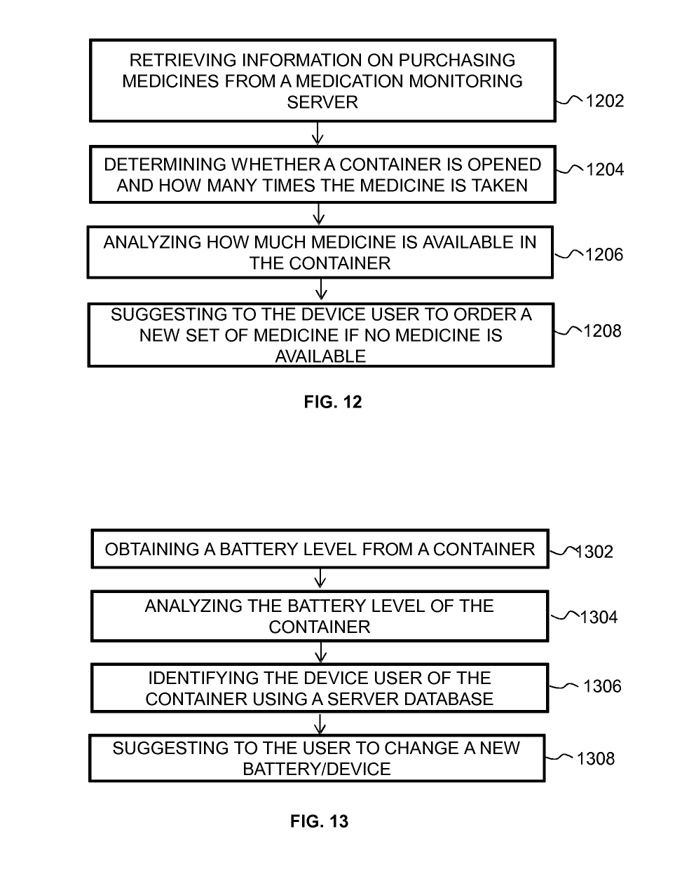

FIG. 12 is a flow diagram that illustrates a method for determining a quantity of medicines available in the container 102 using a mobile device 104, in accordance with an embodiment of the present disclosure. At step 1202, information of medicines are retrieved from a server 108. At step 1204, it is determined whether the container opens and how many times the medicine is taken. At step 1206, the quantity of medicines available in the container 102 is analysed. At step 1208, it is suggested to the device user to order a new set of medicines, when the medicine is no more available in the container 102, or it amount has fallen below a pre-set threshold.

FIG. 13 is a flow diagram that illustrates a method for communicating a battery level of a container 102 to the mobile device 104 from a server 108, in accordance with an embodiment of the present disclosure. At step 1302, a battery level of the container 102 is obtained. At step 1304, the battery level of the container 102 is analysed. At step 1306, the user is identified using a database. At step 1308, changing a new battery or a new container is suggested to the corresponding device user.

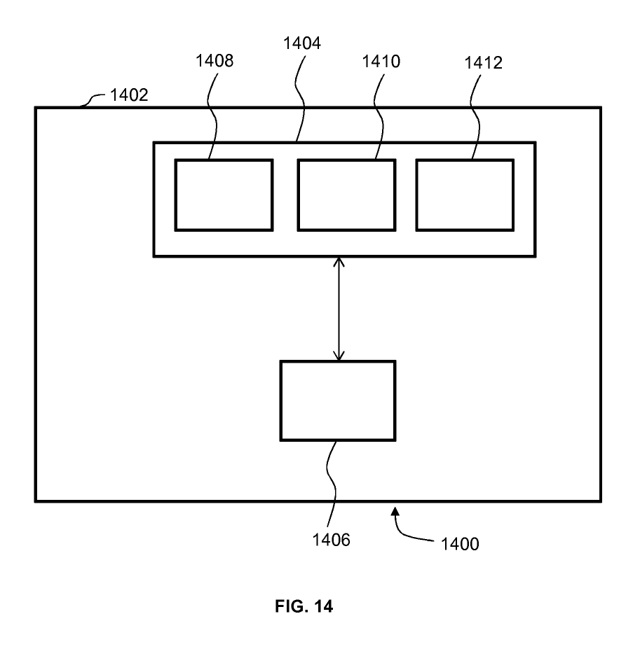

Referring to FIG. 14, illustrated is a functional block diagram 1400 of a container 1402, in accordance with an embodiment of the present disclosure. The functional block diagram 1400 of the container 1402 includes a sensor unit 1404, and a communication unit 1406. The sensor unit 1404 includes a temperature sensor 1408 for measuring temperature inside the container 1402, a humidity sensor 1410 for measuring humidity inside the container 1402, and an acceleration sensor 1412 for measuring accelerations of a cap to identify opening or closing the cap. The sensor unit 1404 is communicatively coupled with the communication unit 1406 to communicate measurement data to the communication unit 1406. The communication unit 1406 communicates the measurement data to a server.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method for managing medication of a user, wherein a container (102) for medicaments comprising a storage chamber and a cap for closing the storage chamber is used, wherein the container comprises
a temperature sensor for measuring temperature inside the container;
a humidity sensor for measuring humidity inside the container;
an accelerometer sensor arranged in connection with the cap;
communication means; and
a first connecting part and a second connecting part arranged to attach to each other via magnetic forces,
the method comprising
pre-setting the magnetic force between the first connecting part and the second connecting part to correspond to a given type of container; and
recognising the type of container by the acceleration of the second connecting part during the attachment of the second connecting part to the first connecting part.

2. A method according to claim 1, wherein the method further comprises
recognising opening and closing of the container by the acceleration of the cap;
calculating the amount of remaining medicine in the container, based on the number of openings of the container;
communicating the amount of remaining medicine to the user;
communicating the temperature and/or humidity inside the container to the user; and
communicating an indication to the user, when the temperature and/or humidity is over and/or under a pre-defined first threshold;
and optionally communicating an order for medicine to a server.

3. A method according to claim 2, wherein the indication to the user comprises a pre-warning that within a pre-defined time, the temperature and/or humidity will be over and/or under the storage temperature and/or humidity of the medicine and/or the indication to the user comprises a warning that the medicine is no longer usable.

4. A method according to claim 1 wherein the method further comprises communicating the amount of remaining medicine and/or communicating the temperature and/or humidity inside the container to a server, and the server providing indications to the user.

5. A method according to claim 1, wherein the first connecting part comprises magnetic material and the second connecting part comprises magnetic material or ferromagnetic material.

6. A method according to claim 1, wherein the first connecting part is arranged in the storage chamber and the second connecting part is arranged in the cap.

7. A method according to claim 1, wherein the first connecting part is arranged in a first part of the cap and the second connecting part is arranged in a second part of the cap, which second part of the cap is arranged in the first part of the cap.

8. A method according to claim 1, wherein the cap comprises a removable component, which component comprises the temperature sensor, the humidity sensor, the accelerometer sensor, the communication means and the second connecting part.
